(19) United States
(12) Patent Application Publication
(10) Pub. No.: US 2009/0023958 A1
(43) Pub. Date: Jan. 22, 2009

(54) Process for preparing dimethyl ether from crude methanol in an adiabatic reactor

(76) Inventors: Ki-won Jun, Daejeon (KR); Hyun-seog Roh, Daejeon (KR); Jae-woo Kim, Chungcheongnam-do (KR); Jeonkeun Oh, Daejeon (KR); Jinhwan Bang, Daejeon (KR)

Correspondence Address: Ronald R. Santucci;Frommer Lawrence & Haug, 745 Fifth Avenue, New York, NY, 10151, US

(21) Appl. No.: 11/665,112

(22) PCT Filed: Aug. 19, 2005
(86) PCT No.: PCT/KR05/02751

§ 371 Date: Apr. 11, 2007

(30) Foreign Application Priority Data
Oct. 15, 2004 (KR) 10-2004-0082721

(51) Int. Cl. C07C 41/09

(52) U.S. Cl. 568/698

(57) ABSTRACT

The present invention relates a process for preparing dimethyl ether from crude methanol in an adiabatic reactor(s)/and more particularly to a process for preparing dimethyl ether wherein crude methanol containing water is dehydrated by using (1) a catalytic system in which the reactant contacts a catalyst 1 of hydrophobic zeolite partially substituted by a specific metal cation and subsequently a catalyst 2 selected from γ-alumina or silica-alumina; and (2) an adiabatic reactor(s). In the present invention, the dehydration is accomplished effectively by avoiding the formation or byproducts and the deactivation of catalyst. Therefore, dimethyl ether useful as clean fuel and a raw material in chemical industry can be produced from crude methanol with an enhanced yield in an adiabatic reactor(s).

### BACKGROUND OF THE INVENTION

1. Field of the Invention

The present invention relates to a process for preparing dimethyl ether from crude methanol in an adiabatic reactor(s), and more particularly to a process for preparing dimethyl ether, wherein crude methanol containing water is dehydrated over a catalytic system which consists of a hydrophobic zeolite catalyst 1 partially substituted with a specific metal cation and a catalyst 2 selected from γ-alumina or silica-alumina, and the reactant sequentially contacts the catalyst 1 and the catalyst 2 in an adiabatic reactor or two adiabatic reactors, which proceeds the dehydration effectively thereby preventing the deactivation of catalysts and the generation of byproducts and produces dimethyl ether useful as clean fuel and a raw material in chemical industry from crude methanol with an enhanced yield.

2. Background of the Related Art

Dimethyl ether (DME) is widely known as an aerosol propellant, a refrigerant, and a useful raw material in chemical industry as well as a clean fuel. Recently, dimethyl ether has drain much of public attention as a next generation energy, source to resolve the drawbacks of natural gas because it discharges incombustible gas of hydrocarbon at an extremely low level. Therefore, it is in urgent need to develop a novel process for preparing dimethyl ether with great efficiency.

In general, dimethyl ether is manufactured in industry by dehydrating methanol as illustrated in the following reaction formula 1.

2CH₃OH→CH₃OCH₃+H₂O (1)

In the process for preparing dimethyl ether, the dehydration of methanol is accomplished at 250-450° C. by using a conventional solid acid catalyst. In detail, γ-alumina [Japanese Patent Laid-open Number 1984-16845], silica-alumina [Japanese Patent Laid-open Number 1984-42333] and the like are usually adopted as a catalyst. However, γ-alumina and silica-alumina can adsorb water easily on the surface due to their hydrophilic property and thus the amount of active sites is decreased resulting in the reduction in the catalytic activity. Accordingly, if methanol as a raw material to prepare dimethyl ether contains water, it causes the solid acid catalyst to lose its catalytic activity remarkably.

To solve the foregoing problems, the water content of methanol in the process for preparing dimethyl ether is generally reduced at less than hundreds of ppm. However, crude methanol manufactured from synthesis gas contains 10-20% of water as a byproduct and thus methanol should be distilled to remove water completely. Further, unreacted methanol is recycled and it can contain a large amount of water remaining after the dehydration and thus the methanol should be distilled to remove water.

Hence, a novel catalyst not easily deactivated by water would be able to greatly reduce energy consumption during the distillation process and improve cost-effectiveness.

The conversion of methanol to dimethyl ether is accomplished by using an acidic catalyst. Since dimethyl ether is an intermediate compound to prepare hydrocarbons, an acid catalyst may vary greatly in its activity and selectivity depending upon the strength of acid sites. In detail, methanol is first converted into dimethyl ether and then finally converted to hydrocarbons in the presence of a catalyst having strong acid sites thereby producing hydrocarbons as byproducts. In contrast, methanol may not be converted sufficiently to dimethyl ether in the presence of a catalyst having weak acid sites due to its relatively low catalytic activity.

Preferably, the acid catalyst resistant to adsorption of water molecules can be hydrophobic zeolite such as USY, mordenite, ZSM series and Beta. Unfortunately, this catalyst may reduce the selectivity due to highly strong acid sites, because it produces hydrocarbons and coke through side reactions. The conventional H-USY, H-ZSM-5 or H-beta zeolites have a drawback that they produce hydrocarbons as byproducts such as methane, ethane and propane due to highly strong add sites. The hydrocarbons as byproducts are low molecular weight alkanes of little value and it also deactivate catalysts by coking.

### SUMMARY OF THE INVENTION

The object of the present invention is to provide a process for preparing dimethyl ether from crude methanol with high yield without producing byproducts by using an adiabatic reactor under a catalytic system.

The present invention has a feature to provide a process for preparing dimethyl ether by dehydrating methanol, wherein the dehydration is performed in an adiabatic reactor(s) by using a catalytic system on which the reactant contacts a catalyst 1 of Formula 1 and subsequently a catalyst 2 selected from γ-alumina or silica-alumina.

HₓM_{(1-x)/n}Z (1)

In the above Formula 1, H is a proton; M is at least one cations selected from metal cations belonging to IA group, IIA group or IB group in Periodic Table; n is an oxidation number of substituted cation (M); x is a proton content in the range of 10-90 mol %; and Z is a hydrophobic zeolite in the range of 20-200 of SiO₂/Al₂O₃ ratio.

### DETAILED DESCRIPTION OF THE PREFERRED EMBODIMENT

Hereinafter, the present invention will be described more clearly as follows.

The present invention provides a process for preparing dimethyl ether, comprising steps of: (1) preparing a catalytic system on which the reactant contacts a catalyst 1 of Formula 1 partially substituted by a metal cation for a proton (H⁺) of hydrophobic zeolite and a catalyst 2 selected from γ-alumina or silica-alumina; (2) packing the catalyst 1 and the catalyst 2 in an adiabatic reactor(s); and (3) dehydrating.

The process for preparing dimethyl ether described above can curtail byproducts including coke and light hydrocarbons such as methane, ethane and propane remarkably and improve the yield of dimethyl ether efficiently. For a raw material, a methanol can be selected in the broad range, because 2 different catalysts are packed in an adiabatic reactor(s) effectively. Preferably, methanol can include crude methanol containing a certain amount of water and traditional pure methanol and more preferably, methanol containing a certain amount of water.

Conventionally, substituted hydrophobic zeolite, γ-alumina and silica-alumina have been used as catalysts to dehydrate methanol in the process for preparing dimethyl ether. However, the catalysts possess problems of the low yield and/or selectivity because they have the hydrophilic property or strong acid sites resulting in decrease in the catalytic activity. In the present invention, a catalytic system employing 2 different kinds of catalysts specified above in series is introduced. In the catalytic system, the ratio of catalysts is adjusted. Further, an adiabatic reactor is adopted instead of a traditional isothermal reactor. When crude methanol is added, temperature, pressure and space velocity are maintained in a specified level. The present invention does not simply coordinate conventional catalysts, but introduces a new reactor and a new catalytic system to improve the process for preparing dimethyl ether.

The process of the present invention for preparing dimethyl ether from crude methanol in an adiabatic reactor will be described more dearly as follows.

The adiabatic reaction is accomplished by blocking heat exchange with an outer environment. The adiabatic reaction is easier and more convenient to design and manufacture a reactor than conventional isothermal reactions and thus reduces the cost to prepare dimethyl ether. However, the adiabatic reaction may be disadvantageous and may provoke an adverse action, if a conventional catalyst such as partially substituted hydrophobic zeolite, γ-alumina or silica-alumina is packed independently in the adiabatic reactor.

That is, the dehydration of methanol is an exothermic reaction to increase the temperature within the reactor by 50-100° C. In detail, if the temperature of entrance is approximately 250° C., the temperature of exit will be approximately 300° C. in the reactor, when methanol containing 20 mol % of water reaches approximately 40-50% of conversion. On the contrary, if γ-alumina or silica-alumina is utilized alone, crude methanol containing water is hardly dehydrated at the temperature of entrance, approximately 250° C. If hydrophobic zeolite is used alone, it results in producing light hydrocarbons.

Accordingly, the present invention has a technical feature that a catalytic system, in which the reactant contacts specific catalysts successively, is packed in the inside of adiabatic reactor, in order to prepare dimethyl ether from crude methanol containing water in an adiabatic reactor efficiently.

The catalytic system of the present invention comprises a catalyst 1 of hydrophobic zeolite partially substituted and a catalyst 2 selected from γ-alumina or silica-alumina and is introduced in the adiabatic reactor. The catalysts will be described more clearly as follows.

Preferably, the catalyst 1 contacting first with methanol may be a hydrophobic zeolite partially substituted by metal cations for protons.

In general, hydrophobic zeolite used to dehydrate methanol can be USY, mordenite, ZSM system and Beta, but problematic to generate byproducts such as light hydrocarbons due to strong acid sites. In the present invention, hydrophobic zeolite partially substituted with metal cations for protons is adopted in order to eliminate the strong acid sites. Preferably, the hydrophobic zeolite can be USY, mordenite, ZSM system, Beta and the like, if partially substituted. Preferably, SiO₂/Al₂O₃ ratio can be adjusted in 20-200. If SiO₂/ Al₂O₃ ratio is less than 20, the catalyst is adsorbed water easily due to the hydrophilic property and deactivated. In contrast, if SiO₂/Al₂O₃ ratio is more than 200, methanol cannot be dehydrated effectively due to the lack of acid sites.

In the present invention, hydrophobic zeolite is adjusted to maintain the proton content (H⁺) in 10-90 mol %. For this purpose, hydrophobic zeolite can be ion-exchanged by using at least one cations belonging to IA group (alkali metal), IIA group (alkaline earth metal), IB group such as Cu and Ag or IIB group such as Zn. Then, the resultant is made to a mixture of cations to control the strength of strong acid sites properly. In detail, H type zeolite having strong acid sites is ion-exchanged by metal cations such as sodium ion and calcium ion to prepare NaH type or CaH type zeolite, adjusting the strength of strong acid sites properly. In practice, there are conventional Na type zeolites such as Na-ZSM-5, Na-Beta and Na-MOR which contains only Na⁺ ions, and H type zeolites such as H-ZSM-5, H-Beta and H-MOR zeolite which contains only H⁺. However, Na type zeolite is ineffective because it retains only weak acid sites. In contrast, H type zeolite is disadvantageous to generate hydrocarbon products because it retains highly strong acid sites.

The substituted hydrophobic zeolite of the present invention may retain acid sites in 10-90 mol % of proton (H⁺) properly.

The process for preparing a hydrophobic zeolite substituted as depicted in Formula 1 will be described more clearly as follows.

In the present invention, the hydrophobic zeolite in 20-200 of SiO₂/Al₂O₃ ratio, such as USY, mordenite, ZSM system and Beta is treated to control the strength of strong acid sites. In detail, it is substituted partially for protons by specific metal cations through an ion exchange method or an impregnation method. Such a substitution may be performed by conventional ion exchange or impregnation methods.

As for the ion exchange method, NH₄ type zeolite is ion-exchanged in a salt solution containing sodium, such as sodium chloride or sodium nitrate solution, followed by drying and calcination in order to prepare NaH type zeolite. As for the impregnation method, NH₄ type zeolite is added to a salt solution containing sodium, such as sodium chloride or sodium nitrate solution and stirred, followed by drying and calcination in order to prepare NaH type zeolite. In order to prepare CaH type zeolite, NH₄ type zeolite is added to a calcium salt solution such as calcium chloride or calcium nitrate solution and stirred, followed by drying and calcination.

The hydrophobic zeolite having 20-200 of SiO₂/Al₂O₃ ratio, such as USY, mordenite, ZSM system and Beta nay vary in the strength of acid sites, depending upon the extent of ion exchange. Preferably, the mole ratio of protons should be maintained in 10-90 mol % in order to prepare a zeolite catalyst having strong acid sites suitable for the present invention.

In case that the catalyst representing as Formula 1 is packed in the upper portion of adiabatic reactor, it can maintain the catalytic activity high without deactivation. Accordingly, the resultant catalyst can perform the dehydration efficiently, even if crude methanol containing water is used as a raw material. Also, the catalyst can suppress the side reaction and curtail byproducts such as hydrocarbons and coke maximally. The catalyst is substituted by metal cations for a part of protons properly to eliminate strong acid sites. This modification may improve the selectivity of dimethyl ether excellently. When the adiabatic reactor is used, the temperature increases by approximately 50-100° C. inside the reactor, because the dehydration of methanol is an exothermic reaction. If the temperature increases highly at more than 300° C., coking and light hydrocarbons may be formed by the side reactions.

Preferably, the adiabatic reactor can be packed in the lower portion by using other kinds of an acid catalyst such as γ-alumina and silica-alumina, weaker than hydrophobic zeolite to prevent the side reactions and to enhance the selectivity of dimethyl ether.

Methanol may react with the catalyst 2, γ-alumina or silica-alumina, right after reacting with the catalyst 1, partially substituted hydrophobic zeolite.

Conventional γ-alumina or silica-alumina tends to adsorb water on the surface due to the hydrophilic property, if solely utilized. Then, it may curtail active sites to decrease the catalytic activity. But, the catalyst has a feature to increase the selectivity of dimethyl ether, when the temperature increases in the reactor due to an exothermic reaction. That is, the hydrophobic zeolite catalyst packed in the upper portion of reactor can hardly escape the side reaction forming coking or light hydrocarbons at a high temperature greater than 300° C. even though strong acid sites are eliminated to some extent by using alkali metal or alkaline earth metal. Therefore, the other acid catalyst weaker than hydrophobic zeolite, selected from γ-alumina and silica-alumina can be used to maintain the selectivity of dimethyl ether at a relatively high temperature. Hence, it is important for the reactant to contact the catalysts 1 and 2 in series. Preferably, the catalyst 1 of hydrophobic zeolite partially substituted should be contacted first and then the catalyst 2 of γ-alumina or silica-alumina later.

The catalysts can be packed in one adiabatic reactor to separate catalyst layers respectively and otherwise, the catalysts can be packed in separate adiabatic reactors to be connected together. Preferably, the catalyst 2 should be maintained in 20-80 volume % with reference to total volume of catalysts. The total volume is measured to combine the volume of hydrophobic zeolite partially substituted and the volume of γ-alumina or silica-alumina. If the catalyst 2 is less than 20 volume %, the reaction may proceed in the lower portion excessively, due to the strong activity of hydrophobic zeolite and thus, produce hydrocarbons as byproducts to reduce the selectivity. In contrast, if the catalyst 2 has more than 80 volume %, the reaction cannot be performed effectively due to the small amount of hydrophobic zeolite.

In the present invention, the catalysts may not be deactivated during the dehydration, since 2 different kinds are packed independently in the adiabatic reactor. Traditional catalysts have a problem that they are easily deactivated during dehydration. Besides, the catalyst of the present invention does not produce hydrocarbons so as to produce dimethyl ether with a high yield. Further, the catalyst packed in the upper portion of reactor can sustain the catalytic activity high for a long time without deactivation and dehydrate crude methanol containing water effectively.

Preferably, the water content of crude methanol is maintained in 5-50 mol %. If the water content is less than 5 mol %, methanol as a raw material may not be economical to consume much energy during the purification. In contrast, if the water content is more than 50 mol %, the effective amount of reactant becomes too small. As a result, the above-mentioned range should be maintained to maximize the effect.

In the meantime, the process of the present invention for preparing dimethyl ether from crude methanol containing water is accomplished by the same procedure with the traditional process, but using a catalytic system wherein the reactant contacts a hydrophobic zeolite 1 partially substituted and subsequently γ-alumina or silica-alumina catalyst 2 is packed within the adiabatic reactor.

In a preferred embodiment of the present invention, one adiabatic reaction is employed to pack hydrophobic zeolite 1 partially substituted in the upper portion and γ-alumina or silica-alumina catalyst 2 in the lower portion. Then, methanol is passed from the upper portion to the lower portion sequentially to prepare dimethyl ether.

In order to remove moisture and contaminants adsorbed on the catalyst, the upper portion and the lower portion are pretreated respectively. Precisely, an inert gas such as nitrogen can be flowed at 200-350° C. and a flow rate of 20-100 ml/g-catalyst/min.

Methanol is passed through the catalysts preheated by the above-mentioned procedure in the reactor. Preferably, the temperature of entrance can be maintained at 150-400° C. in the reactor. If the temperature of entrance is less than 150° C., the conversion is reduced due to low reaction rate. In contrast, if the temperature is more than 400° C., the production of dimethyl ether is unfavorable thermodynamically to provoke side reactions. Preferably, the reaction pressure can be maintained at 1-100 atm. If the pressure is more than 100 atm, the reaction cannot be operated properly. Preferably, liquid hourly space velocity (LHSV) can be maintained in 0.05-50 h⁻¹ with reference to pure methanol to proceed the dehydration. If LHSV value is less than 0.05 h⁻¹, the productivity of reaction becomes too low. In contrast, if LHSV value is more than 50 h⁻¹, the contact time on catalyst is so short that the conversion is reduced.

The effective catalytic system of the present invention can be also a connection of two reactors, which are packed with catalysts 1 and 2 respectively. However, the order of methanol contacting catalysts should be considered as an important factor.

As described above, the adiabatic reactor of the present invention should be packed with the hydrophobic zeolite catalyst of formula 1 in the upper portion and the γ-alumina catalyst in the lower portion independently. As a result, crude methanol containing 5-50 mol % of water as a raw material is observed not to deactivate the catalyst even after the dehydration. Further, dimethyl ether is produced width a high yield without byproducts such as hydrocarbons.

Practical and presently preferred embodiments of the present invention are illustrated as shown in the following Examples. However, it will be appreciated that those skilled in the art, on consideration of this disclosure, may make modifications and improvements within the spirit and scope of the present invention.

### EXAMPLE 1

(A) Preparation of a Hydrophobic Zeolite Catalyst

NH₄-ZSM-5(SiO₂/Al₂O₃=30) zeolite was added to 0.1 N of NaCl solution, stirred at 80° C. for 24 hours and then washed out by using distilled water. After that, the resulting zeolite was dried at 120° C. for 12 hours and calcined at 500° C. for 12 hours to produce NaH-ZSM-5 (ion exchange rate of Na=44 mol %). A paste was prepared by adding 2.5% nitric acid (nitric acid solution/zeolite wt. ratio=0.8) to boehmite (boehmite/zeolite wt. ratio=1). To this paste, the above NaH-ZSM-5 zeolite particles and water (water/zeolite wt. ratio=0.5) were added, kneaded and then extruded. The extrudate was dried at 120° C. for 12 hours and calcined at 600° C. for 6 hours to obtain a catalyst. The boehmite ingredient in the catalyst is converted into gamma-alumina during the calcination at 600° C. for 6 hours.

(B) Placement of Catalysts in an Adiabatic Reactor

100 ml of γ-alumina catalyst was packed in the lower portion of an adiabatic reactor and then 100 ml of the hydrophobic zeolite prepared in above-mentioned stage (A) was packed in the upper portion of the same adiabatic reactor.

Next, methanol containing 20 mol % of water was passed through the catalyst layers at LHSV of 7.0 h⁻¹ and under 10 atm. The temperature at the reactor inlet was controlled to be kept at 230° C. and the reactant was made to contact the catalyst layer of hydrophobic zeolite, and then the catalyst layer of γ-alumina, thereby converting it into dimethyl ether. As a result, the yield of dimethyl ether prepared above and the yield of hydrocarbons as byproducts were measured and the data are illustrated in Table 1.

### EXAMPLE 2

(A) Preparation of a Hydrophobic Zeolite Catalyst

H-ZSM-5(SiO₂/Al₂O₃=40) zeolite was impregnated with aqueous sodium nitrate containing Na⁺ equivalent to 50 mole % of H⁺ in the zeolite. A paste was prepared by adding 2.5% nitric acid (nitric acid solution/zeolite wt. ratio=0.8) to boehmite (boehmite/zeolite wt. ratio=1). To this paste, the above NaH-ZSM-5 zeolite particles and water (water/zeolite wt. ratio=0.5) were added, kneaded and then extruded. The extrudate was dried at 120° C. for 12 hours and calcined at 600° C. for 6 hours to obtain a catalyst.

(B) Placement of Catalysts in an Adiabatic Reactor

The catalyst was packed in an adiabatic reactor by the same procedure described in Example 1.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

### EXAMPLE 3

(A) Preparation of a Hydrophobic Zeolite Catalyst

To H-ZSM-5(SiO₂/Al₂O₃=40) zeolite aqueous sodium nitrate containing Na⁺ equivalent to 30 mole % of H⁺ in the zeolite and Kaolin were added at 20 wt %, kneaded and then extruded. After that, the resultant was dried at 120° C. for 12 hours and calcined at 600° C. for 6 hours. As a result, a catalyst in an extrudate form was prepared.

(B) Placement of Catalysts in an Adiabatic Reactor

The catalyst was packed in an adiabatic reactor by the same procedure described in Example 1.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

### EXAMPLE 4

(A) Preparation of a Hydrophobic Zeolite Catalyst

To H-ZSM-5(SiO₂/Al₂O₃=40) zeolite aqueous sodium nitrate containing Na⁺ equivalent to 40 mole % of H⁺ in the zeolite and Kaolin were added at 20 wt %, kneaded and then extruded. After that, the resultant was dried at 120° C. for 12 hours and calcined at 600° C. for 6 hours. As a result, a catalyst in an extrudate form was prepared.

(B) Placement of Catalysts in an Adiabatic Reactor

The catalyst was packed in an adiabatic reactor by the same procedure described in Example 1.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

### EXAMPLE 5

(A) Preparation of a Hydrophobic Zeolite Catalyst

H-ZSM-5(SiO₂/Al₂O=40) zeolite was impregnated with aqueous potassium nitrate containing K³⁰ equivalent to 50 mole % of H⁺ in the zeolite. A paste was prepared by adding 2.5% nitric acid (nitric acid solution/zeolite wt. ratio=0.8) to boehmite (boehmite/zeolite wt. ratio=1). To this paste, the above NaH-ZSM-5 zeolite particles and water (water/zeolite wt. ratio=0.5) were added, kneaded and then extruded. The extrudate was dried at 120° C. for 12 hours and calcined at 600° C. for 6 hours to obtain a catalyst.

(B) Placement of Catalysts in an Adiabatic Reactor

80 ml of γ-alumina catalyst was packed in the lower portion of an adiabatic reactor and then 120 ml of the hydrophobic zeolite prepared in above-mentioned stage (A) was packed in the upper portion of the same adiabatic reactor.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

### EXAMPLE 6

(A) Preparation of a Hydrophobic Zeolite Catalyst

H-ZSM-5(SiO₂/Al₂O₃=40) zeolite was impregnated with aqueous cesium nitrate containing Ce⁺⁺ equivalent to 50 mole % of H⁺ in the zeolite. A paste was prepared by adding 2.5% nitric acid (nitric acid solution/zeolite wt. ratio=0.8) to boehmite (boehmite/zeolite wt. ratio=1). To this paste, the above CeH-ZSM-5 zeolite particles and water (water/zeolite wt. ratio=0.5) were added, kneaded and then extruded. The extrudate was dried at 120° C. for 12 hours and calcined at 600° C. for 6 hours to obtain a catalyst.

(B) Placement of Catalysts in an Adiabatic Reactor

80 ml of γ-alumina catalyst was packed in the lower portion of an adiabatic reactor and then 120 ml of the hydrophobic zeolite prepared in above-mentioned stage (A) was packed in the upper portion of the same adiabatic reactor.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

### EXAMPLE 7

(A) Preparation of a Hydrophobic Zeolite Catalyst

H-ZSM-5(SiO₂/Al₂O₃=40) zeolite was impregnated with aqueous magnesium nitrate containing Mg⁺⁺ equivalent to 50 mole % of H⁺ in the zeolite. A paste was prepared by adding 2.5% nitric acid (nitric acid solution/zeolite wt. ratio=0.8) to boehmite (boehmite/zeolite wt. ratio=1). To this paste, the above MgH-ZSM-5 zeolite particles and water (water/zeolite wt. ratio=0.5) were added, kneaded and then extruded. The extrudate was dried at 120° C. for 12 hours and calcined at 600° C. for 6 hours to obtain a catalyst.

(B) Placement of Catalysts in an Adiabatic Reactor

120 ml of γ-alumina catalyst was packed in the lower portion of an adiabatic reactor and then 80 ml of the hydrophobic zeolite prepared in above-mentioned stage (A) was packed in the upper portion of the same adiabatic reactor.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

### EXAMPLE 8

(A) Preparation of a Hydrophobic Zeolite Catalyst

H-ZSM-5(SiO₂/Al₂O₃=40) zeolite was impregnated with aqueous calcium nitrate containing Ca⁺⁺ equivalent to 50 mole % of H⁺ in the zeolite. A paste was prepared by adding 2.5% nitric acid (nitric acid solution/zeolite wt. ratio=0.8) to boehmite (boehmite/zeolite wt. ratio=1). To this paste, the above CaH-ZSM-5 zeolite particles and water (water/zeolite wt. ratio=0.5) were added, kneaded and then extruded. The extrudate was dried at 120° C. for 12 hours and calcined at 600° C. for 6 hours to obtain a catalyst.

(B) Placement of Catalysts in an Adiabatic Reactor

120 ml of γ-alumina catalyst was packed in the lower portion of an adiabatic reactor and then 80 ml of the hydrophobic zeolite prepared in above-mentioned stage (A) was packed in the upper portion of the same adiabatic reactor.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

### EXAMPLE 9

(A) Preparation of a Hydrophobic Zeolite Catalyst

NH-ZSM₄-ZSM-5(SiO₂/Al₂O₃=30) zeolite was added to 0.5 N of cupric nitrate solution, stirred at 80° C. for 24 hours and then washed out by using distilled water. The resultant powder was again added to 0.5 N of cupric nitrate solution, stilled at 80° C. for 24 hours and then washed out bar using distilled water. After that, the resultant zeolite was dried at 120° C. for 12 hours and calcined at 500° C. for 12 hours to produce CuH-ZSM-5 (ion exchange rate of Cu=80 mol %). Kaolin was added to CuH-ZSM-5 mentioned above at 40 wt %, kneaded and then extruded. The extrudate was dried at 120° C. for 12 hours and calcined at 600° C. for 6 hours to obtain a catalyst.

(B) Placement of Catalysts in an Adiabatic Reactor

100 ml of γ-alumina catalyst was packed in the lower portion of an adiabatic reactor and then 100 ml of the hydrophobic zeolite prepared in above-mentioned stage (A) was packed in the upper portion of the same adiabatic reactor.

Next, methanol was dehydrated to prepare dimethyl ether by the same methods described in Example 1, according to stage (A) and stage (B), but methanol containing 30% of water was fed as a reactant. As a result, the yield of dimethyl ether prepared above and the yield of hydrocarbons as byproducts were measured and the data are illustrated in Table 1.

### EXAMPLE 10

(A) Preparation of a Hydrophobic Zeolite Catalyst

NH₄-ZSM-5(SiO₂/Al₂O₃=30) zeolite was added to 0.5 N of zinc nitrate solution, stirred at 80° C. for 24 hours and then washed out by using distilled water. The resultant powder was again added to 0.5 N of zinc nitrate solution, stirred at 80° C. for 24 hours and then washed out by using distilled water. After that, the resultant zeolite was dried at 120° C. for 12 hours and calcined at 500° C. for 12 hours to produce ZnH-ZSM-5 (ion exchange rate of Zn=77 mol %). Kaolin was added to ZnH-ZSM-5 mentioned above at 40 wt %, kneaded and then extruded. The extrudate was dried at 120° C. for 12 hours and calcined at 600° C. for 6 hours to obtain a catalyst.

(B) Placement of Catalysts in an Adiabatic Reactor

100 ml of γ-alumina catalyst was packed in the lower portion of an adiabatic reactor and then 100 ml of the hydrophobic zeolite prepared in above-mentioned stage (A) was packed in the upper portion of the same adiabatic reactor.

Next, methanol was dehydrated to prepare dimethyl ether by the same method described in Example 1, according to stage (A) and stage (B), but methanol containing 30% of water was fed as a reactant. As a result, the yield of dimethyl ether prepared above and the yield of hydrocarbons as byproducts were measured and the data are illustrated in Table 1.

### EXAMPLE 11

The same procedure described in Example 1 was carried out to prepare catalyst and dehydrate methanol, but the temperature of reactant at the inlet of reactor, the LHSV and the reaction pressure were 250° C., 7.0 h⁻¹ and 10 atm, respectively. As a result, the yield of dimethyl ether prepared above and the yield of hydrocarbons as byproducts were measured and the data are illustrated in Table 1.

### EXAMPLE 12

The same procedure described in Example 1 was carried out to prepare catalyst and dehydrate methanol, but 100 ml of silica-alumina (silica: 86 wt %) was packed in the lower portion of adiabatic reactor instead of γ-alumina. As a result, the yield of dimethyl ether prepared above and the yield of hydrocarbons as byproducts were measured and the data are illustrated in Table 1.

### EXAMPLE 13

The scene procedure described in Example 1 was carried out to prepare catalyst and dehydrate methanol, but methanol containing 30 mol % of water was passed through the catalyst layers at LHSV of 7.0 ⁻¹. As a result, the yield of dimethyl ether prepared above and the yield of hydrocarbons as byproducts were measured and the data are illustrated in Table 1 as follows.

### COMPARATIVE EXAMPLE 1

The same procedure described in Example 1 was carried out to dehydrate methanol, but 200 ml of γ-alumina catalyst only was packed in an adiabatic reactor and methanol containing 30 mol % of water was passed through the single catalyst layer. As a result, the yield of dimethyl ether prepared above and the yield of hydrocarbons as byproducts were measured and the data are illustrated in Table 1.

### COMPARATIVE EXAMPLE 2

The same procedure described in Example 1 was carried out to dehydrate methanol, but 200 ml of silica-alumina (silica: 86 wt %) catalyst only was packed in an adiabatic reactor and the reactant was passed through the single catalyst layer. As a result, the yield of dimethyl ether prepared above and the yield of hydrocarbons as byproducts were measured and the data are illustrated in Table 1.

### COMPARATIVE EXAMPLE 3

The same procedure described in Example 1 is carried out to prepare catalyst and dehydrate methanol, but 200 ml of hydrophobic zeolite catalyst (NaH-ZSM-5) only was packed in an adiabatic reactor and the reactant was passed through the single catalyst layer. As a result, the yield of dimethyl ether prepared above and the yield of hydrocarbons as byproducts were measured and the data are illustrated in Table 1.

### COMPARATIVE EXAMPLE 4

(A) Preparation of a Hydrophobic Zeolite Catalyst

To NH₄-ZSM-5(SiO₂/Al₂O₃=40) zeolite/boehmite (boehmite/zeolite wt. ratio=1), 2.5% nitric acid (nitric acid solution/zeolite wt. ratio=0.8) and water (water/zeolite wt. ratio=0.5) were added, kneaded and then extruded. After that, the resultant extrudate was dried at 120° C. for 12 hours and calcined at 600° C. for 6 hours. As a result, a catalyst in an extrudate form was prepared.

(B) Placement of Catalysts in an Adiabatic Reactor

The catalyst was packed in an adiabatic reactor by the same procedure described in Example 1.

Next, methanol was dehydrated to prepare dimethyl ether by the same methods described in Example 1, according to stage (A) and stage (B). As a result, the yield of dimethyl ether prepared above and the yield of hydrocarbons as byproducts were measured and the data are illustrated in Table 1.

In Table 1, the results obtained by the procedure using catalysts and reaction conditions for dehydrating methanol as described in Example 1-13 and Comparative Example 1-4, are summarized as follows.

TABLE 1

| | Catalyst | | Water | Productivity (%) | |
|---|---|---|---|---|---|
| | | lower | content | dimethyl | hydro- |
| Item | upper portion | portion | (mol %) | ether | carbon |
| Example 1 | NaH-ZSM-5 | γ-alumina | 20 | 80.5 | 0.0 |
| Example 2 | NaH-ZSM-5 | γ-alumina | 20 | 80.4 | 0.0 |
| Example 3 | NaH-ZSM-5 | γ-alumina | 20 | 79.3 | 0.0 |
| Example 4 | NaH-ZSM-5 | γ-alumina | 20 | 78.1 | 0.0 |
| Example 5 | KH-ZSM-5 | γ-alumina | 20 | 77.6 | 0.0 |
| Example 6 | CsH-ZSM-5 | γ-alumina | 20 | 77.2 | 0.0 |
| Example 7 | MgH-ZSM-5 | γ-alumina | 20 | 76.3 | 0.0 |
| Example 8 | CaH-ZSM-5 | γ-alumina | 20 | 76.6 | 0.0 |
| Example 9 | CuH-ZSM-5 | γ-alumina | 30 | 74.5 | 0.0 |
| Example 10 | ZnH-ZSM-5 | γ-alumina | 30 | 74.1 | 0.0 |
| Example 11 | NaH-ZSM-5 | γ-alumina | 20 | 75.3 | 0.0 |
| Example 12 | NaH-ZSM-5 | silica- | 20 | 72.6 | 0.0 |
| | | alumina | | | |
| Example 13 | NaH-ZSM-5 | γ-alumina | 30 | 75.2 | 0.0 |
| Comparative | γ-alumina | | 30 | 1.2 | 0.0 |
| Example 1 | | | | | |
| Comparative | silica-alumina | | 20 | 0.6 | 0.0 |
| Example 2 | | | | | |
| Comparative | NaH-ZSM-5 | | 20 | 76.3 | 4.7 |
| Example 3 | | | | | |
| Comparative | H-ZSM-5 | γ-alumina | 20 | 72.1 | 6.1 |
| Example 4 | | | | | |

As illustrated in Table 1, it is clarified that dimethyl ether in Example 1-13 is prepared with higher yields, compared with dimethyl ether in Comparative Example 1-2 using traditional catalysts. In the present invention, methanol is dehydrated by passing through the catalyst 1 of hydrophobic zeolite partially substituted by specific metal cations for a pair of protons and the catalyst 2 selected from γ-alumina or silica-alumina subsequently in the adiabatic reactor to prepare dimethyl ether. Thereby, the process for preparing dimethyl ether of the present invention does not generate hydrocarbons as byproducts at all even at a high yield, in contrast to Comparative Example 3 employing hydrophobic zeolite solely in an adiabatic reactor and Comparative Example 4 employing H type hydrophobic zeolite non-substituted by a metal cation and γ-alumina.

In detail, methanol containing 30 mol % of water as a raw material is dehydrated by using the most conventional catalyst, γ-alumina in Comparative Example 1, but dimethyl ether is produced with a low yield, approximately 1% due to the deactivation of catalyst. Besides, only NaH-ZSM-5 catalyst is packed in the adiabatic reactor in Comparative Example 3, but hydrocarbons are produced in about 5% by the side reaction though dimethyl ether was produced with a relatively high yield, approximately 76%. In this case, the adiabatic reactor continues to increase the temperature in the inside, because all the parts in the reactor are filled with the hydrophobic zeolite. As a result, hydrocarbon products may be generated at a high temperature, as the exit temperature of reactor reaches up to higher than 320° C. In case that H-ZSM-5 zeolite retaining strong acid sites is added to the upper portion of adiabatic reactor as shown in Comparative Example 4, hydrocarbons are generated though dimethyl ether is produced with a high yield. Hydrocarbons generated as byproducts are low molecular weight alkanes, worthless and deactivate catalysts due to the formation of coke.

Therefore, it is confirmed that the process for preparing dimethyl ether of the present invention, comprising steps: (1) adopting crude methanol containing 20-30 mol % of water; (2) employing serially 2 different kinds of catalyst in the upper portion and the lower portion of the reactor respectively; and (3) dehydrating, may improve the yield of dimethyl ether excellently and seldom generates hydrocarbons as byproducts.

As demonstrated above, the catalytic system, wherein the catalyst 1 of hydrophobic zeolite partially substituted by metal cations for protons (H⁺) to adjust the strength of acid sites; and the catalyst 2 selected from γ-alumina or silica-alumina are packed in the adiabatic reactor to dehydrate methanol gives results of no deactivation of catalysts, no formation of hydrocarbon products and increased yield of dimethyl ether by maintaining the catalytic activity high.

The adiabatic reactor adopted in the present invention can reduce the cost since it is easily designed and conveniently manufactured. Besides, even if crude methanol containing water is used, the performance and the stability of catalyst can be attained in a high level. The selectivity of dimethyl ether is also maintained highly. Furthermore, catalysts can be packed effectively by a simple process.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A process for preparing dimethyl ether by dehydrating crude methanol in (an) adiabatic reactor(s), wherein said dehydration is performed over a catalytic system which consists of a catalyst 1 of Formula 1 partially substituted by metal cations for protons (H⁺) of hydrophobic zeolite; and a catalyst 2 selected from γ-alumina and silica-alumina, wherein methanol contacts with the catalyst 1 and the catalyst 2 sequentially,
H_{K}M_{(1-x)/n}Z (1)
wherein H is a proton; M is at least one cation selected from metal cations belonging to IA group, IIA group, IB group or IIB group in Periodic Table; n is the oxidation number of substituted cations (M); x is a proton content in the range of 10-90 mol %; and Z is a hydrophobic zeolite wherein SiO₂/Al₂O₃ ratio is in the range of 20-200 of SiO₂/Al₂O₃.

2. (canceled)

3. The process for preparing dimethyl ether according to claim 1, wherein said adiabatic reactor is so packed with said catalyst 1 and said catalyst 2 that each catalyst has its own layer in a single reactor.

4. The process for preparing dimethyl ether according to claim 1, wherein said adiabatic reactors are two and separately packed with said catalyst 1 and said catalyst 2.

5. (canceled)

6. The process for preparing dimethyl ether according to claim 1, wherein said crude methanol contains 5-50 mol % of water.

7. The process for preparing dimethyl ether according to claim 1, wherein said partially substituted catalyst 1 of hydrophobic zeolite is prepared by partial substitution of at least one metal cations selected from IA group, IIA group, IB group and IIB group for protons with addition of transition alumina and (a) salts of IA group, IIA group, IB group and IIB group metals.

8. The process for preparing dimethyl ether according to claim 1, wherein said catalyst 1 of hydrophobic zeolite is prepared by partial substitution at least one metal cations selected from IA group, IIA group, IB group and IIB group for protons with addition of clay containing at least one metal cations belonging to IA group or IIA group; or with a mixture of a metal salt of at least one metal cations belonging to IA group, IIA group, IB group or IIB group and clay.

9. The process for preparing dimethyl ether according to claim 1, wherein said dehydration is performed at 150-400° C. under 1-100 atm of reaction pressure and at 0.05-50 h⁻¹ of LHSV (liquid hourly space velocity).
